(19) United States
(12) Patent Application Publication
(10) Pub. No.: US 2021/0147891 A1
(43) Pub. Date: May 20, 2021

(54) SUGAR SOLUTION EXHIBITING LITTLE DISCOLOURATION, AND PRODUCTION METHOD THEREFOR

(71) Applicant: KIKKOMAN CORPORATION, Chiba (JP)

(72) Inventors: Kentaro TAN, Chiba (JP); Shigeki TANIZAWA, Chiba (JP)

(21) Appl. No.: 16/765,259

(22) PCT Filed: Nov. 14, 2018
(86) PCT No.: PCT/JP2018/042092

§ 371 Date: May 19, 2020

(30) Foreign Application Priority Data
Nov. 20, 2017 (JP) 2017-222842

(51) Int. Cl. C12P 19/14; C12G 3/08; A23L 27/00; A23L 27/30

(52) U.S. Cl. CPC C12P 19/14; C12G 3/08; A23L 27/33; A23L 27/88; A23L 27/80

(57) ABSTRACT

The purpose of the present invention is to provide, as a sugar solution which has a high amount of extract components, and a low amount of soluble components other than sugar components among the extract components, in comparison to conventional sugar solutions, a reaction product of rice and amylase, or a substance which can be easily obtained from said reaction product. Provided is a sugar solution comprising water, and a reaction product of rice and α amylase provided in said water, wherein the amount of soluble protein in the sugar solution is 200 μg/ml or lower, and the amount of extract components in the sugar solution is at least 43 wt % of the whole sugar solution, with the caveat that: the reaction by which the reaction product of the rice and the α amylase is obtained is a reaction in which water vapour is blown into a liquefied solution or an emulsified solution obtained from rice, α amylase, and water, and is carried out at a temperature of 90-150° C. for a time of 1-3.5 minutes; the liquefication time when obtaining the liquefied solution or the emulsified solution from the rice, the α amylase, and the water is in the range of 15 seconds to 30 minutes; and the liquefication temperature is in the range of 55-80° C.

### TECHNICAL FIELD

The present invention relates to a sugar solution prepared using rice as a raw material, in which the amount of soluble protein is small and the amount of sugar in the extract component is large, and a seasoning or a food and a drink using the sugar solution, and a method for producing the sugar solution.

### BACKGROUND ART

Sugar solutions used as a raw material for producing seasonings and food and drink are also called a saccharified solution, and are produced by liquefying and saccharifying a starch-containing material. For example, to prepare a sugar solution used for producing mirin, a Japanese sweet cooking wine, usually rice grains are liquified and saccharified in the presence of enzyme in a tank by heating and stirring a liquid containing the rice grains and the enzyme, while circulating the liquid by using a pump.

Details of such sugar solutions, including methods for preparation therefor, have been reported (Patent Literatures 1 to 6).

Patent Literature 1 discloses a method for producing a saccharified solution of rice, in which a mixture of raw rice and water is subjected to a high temperature high pressure treatment and then an enzymatic reaction at 45 to 50° C.

Patent Literature 2 discloses a method for producing a high concentration sugar solution using starch, in which starch, a liquefying enzyme and water in an amount corresponding to a high concentration saccharified solution are kneaded and heated to be treated with amylase.

Patent Literature 3 discloses a saccharified solution of grains produced by performing a two-stage liquefaction in which a liquefying enzyme is added and then saccharifying the resultant.

Patent Literature 4 discloses a method for producing starch syrup, in which both precipitation produced by acid treatment and precipitation produced by alkali treatment are removed from a saccharified solution.

Patent Literature 5 discloses a method for preparing starch syrup from broken rice powder, in which enzymatic liquefaction is performed at a relatively high temperature for a short time.

Patent Literature 6 discloses a saccharified solution of rice having a specific amino acid concentration, and a method for producing the same.

Patent Literature 7 discloses a method for producing a sweetener, in which raw material grains which have been heated and pressurized with introducing vapor are directly subjected to a step of fermentation.

Patent Literatures 8 to 10 disclose a method for producing mirin, and the like. Patent Literature 11 also reports techniques related to a sugar solution and a method for producing the same.

When rice is used as a starch-containing material, substances other than starch as well as starch in rice are decomposed and mixed in the sugar solution as a soluble component in the process of producing the sugar solution. Such soluble components are unnecessary for final products, i.e., seasonings and food and drink, and thus a smaller amount of soluble components in a sugar solution improves the yield of products, and is preferable in terms of production efficiency and cost. In the case of mirin, for example, soluble components in the extract component other than sugar, such as soluble protein, need to be removed from unprocessed mirin or mirin produced, as a sediment (precipitation). However, conventional art which produces sugar solutions using rice as a raw material is intended to produce an extract component containing a large amount of sugar efficiently, and reducing the amount of soluble components other than sugar has not been fully investigated.

### PRIOR ART DOCUMENTS

### Patent Document

[Patent Document 1] Japanese Patent No. 3655880
[Patent Document 2] JP 6-261781 A
[Patent Document 3] JP 4-218361 A
[Patent Document 4] JP 4-131051 A
[Patent Document 5] JP 46-5781 B
[Patent Document 6] JP 2014-180249 A
[Patent Document 7] JP 7-255454 A
[Patent Document 8] JP 56-061973 A
[Patent Document 9] JP 03-195472 A
[Patent Document 10] JP 2001-169746 A
[Patent Document 11] Japanese Patent Application No. 2016-101547

### SUMMARY OF INVENTION

### Technical Problem

The present inventors have assumed that a sugar solution composed of a reaction product of rice and amylase itself and water, a reaction medium, in which the amount of a protein component, i.e., the main constituent of soluble components in the extract component other than sugar, is smaller and the amount of sugar in the extract component is larger than those in conventional sugar solutions, enables more efficient production of seasonings or food and drink, and the sugar solution might be suitable for the production.

No sugar solution prepared using rice as a raw material has been obtained before, which is composed of a reaction product of rice and amylase, and water, a reaction medium, in which the amount of soluble components other than sugar in the extract component is small and the amount of sugar in the extract component is large. In current methods for producing sugar solutions, materials need to be mixed several times at intervals, and additional steps of, for example, concentration, is necessary. Thus, those methods cannot give a reaction product in which the amount of soluble components in the extract component other than sugar is small and the amount of sugar in the extract component is large. If a larger amount of amylolytic enzyme is used than usual, the amount of soluble components in the extract component other than sugar and the amount of sugar in the extract component may be modified, but such attempts cannot be deemed to have been thoroughly done presumably because how much they can be modified by increasing the amount of amylolytic enzyme is unknown, and because amylolytic enzyme is expensive.

An object of the present invention is to provide a sugar solution comprising a reaction product of rice and amylase, and water, i.e., a reaction medium, in which the amount of soluble components in the extract component other than sugar is smaller and the amount of sugar in the extract component is larger than those in conventional sugar solutions, and a sugar solution which is easily prepared from a material containing the reaction product.

### Solution to Problem

In view of the above object, the present inventors have found that a sugar solution prepared using rice as a raw material, in which the amount of a soluble component in the extract component other than sugar is significantly smaller than the amount of sugar in the extract component might solve the above problem, and have conducted further studies and as a result have completed the present invention.

The present invention relates to at least the following inventions:

[1] A sugar solution comprising water and a reaction product of rice and a amylase in the water, wherein the amount of soluble protein in the sugar solution is 200 μg/ml or less and the amount of an extract component in the sugar solution is 43% by weight or more based on the total amount of the sugar solution, and wherein

a reaction for preparing the reaction product of rice and α amylase is a reaction performed at a temperature of 90° C. to 150° C. for 1 minute to 3.5 minutes, with blowing water vapor into a liquefied solution or an emulsified solution prepared from rice, α amylase and water, and the time of liquefaction for preparing the liquefied solution or the emulsified solution from rice, α amylase and water is 15 seconds to 30 minutes and the temperature of liquefaction is 55° C. to 80° C.

[2] A sugar solution prepared by further concentrating and/or storing the sugar solution according to [1], wherein the amount of soluble protein in the sugar solution is 200 μg/ml or less and the amount of an extract component in the sugar solution is 43% by weight or more based on the total amount of the sugar solution, and the concentrating is concentrating to a volume of not less than 90% of the volume of the sugar solution before concentration and the storing is storing at 55° C. to 70° C. for 20 minutes to 22 hours.

[3] The sugar solution according to [1] or [2] above, wherein the rice comprises non-glutinous rice (uruchi-mai) flour.

[4] The sugar solution according to any one of [1] to [3] above, wherein the sugar solution is used for producing a seasoning, or a food and a drink.

[5] The sugar solution according to [4] above, wherein the seasoning or a food and a drink is mirin.

[6] A seasoning or a food and a drink, prepared by mixing the sugar solution according to any one of [1] to [4] above.

[7] The seasoning or food and drink according to [6] above, wherein the seasoning or food and drink is mirin.

[8] A method for producing a sugar solution, comprising performing a reaction at a temperature of about 90° C. to about 150° C. for about 1 minute to about 3.5 minutes, with blowing water vapor into a liquefied solution or an emulsified solution prepared from rice, amylase and water to give a reaction product as a sugar solution, in which the amount of soluble protein is 200 μg/ml or less and the amount of an extract component is 43% by weight or more based on the total amount,

wherein the time of liquefaction for preparing the liquefied solution or the emulsified solution from rice, amylase and water is 15 seconds to 30 minutes and the temperature of liquefaction is 55° C. to 80° C.

[9] A raw material for the sugar solution according to any one of [1] to [5] above, in which the raw material is a reaction product of rice and amylase, wherein the value of (the amount of the extract component in the reaction product [% by weight])/(the amount of the soluble protein in the reaction product [w/v %]) is 2,000 or more.

[10] A sugar solution in which the amount of soluble protein is 200 μg/ml or less and the amount of an extract component is 43% by weight or more based on the total amount, and the volume of a sediment before removing the sediment is 35 or less, wherein the volume of the sediment is a ratio of the height of the sediment to the height to the surface of the solution (%), the sediment being produced by pouring the sugar solution into a container having a uniform shape in a longitudinal direction, together with an aggregate, and stirring the mixture and then allowing the mixture to stand indoors for 3 days.

Removal of a sediment is the operation of removing a sediment, which is a fine solid formed when a sugar solution is pasteurized. The operation means compression by a press filter or natural separation in a clarification tank.

### Advantageous Effects of Invention

The present invention provides a sugar solution in the form of a reaction product of rice and α amylase, in which the amount of soluble protein, i.e., the main constituent of soluble components other than sugar, is small and the amount of sugar in the extract component is large, and which is useful for a seasoning and food and drink. Thus, the present invention enables more efficient production of a seasoning or a food and a drink using a sugar solution. The present invention also provides a seasoning and food and drink using the sugar solution.

The present invention also provides a method for producing a sugar solution using a reaction product of rice and α amylase as an extract component, in which the amount of soluble protein is small and the amount of sugar in the extract component is large. The method for producing a sugar solution of the present invention is significantly different from conventional methods in that the present method adopts a combination of a temperature and a time most suitable for performing an enzymatic reaction in a short time.

Furthermore, using the sugar solution of the present invention for food and drink or seasonings can create a taste and flavor similar to those of conventional food and drink and seasonings. Having a taste and flavor unchanged from those of conventional food and drink and seasonings is important particularly for the same product sold for a long period, such as mirin.

Furthermore, discoloration of the sugar solution of the present invention is much smaller than that of conventional sugar solutions, in particular, those concentrated or produced by heating long time. Thus, the sugar solution of the present invention also has an advantageous effect of allowing for more flexibility in the color of products using the sugar solution and not reducing the value of products due to unnecessary discoloration.

The above difference between the present invention and conventional art is at least in the amount of sugar and the amount of soluble protein in the extract component. Meanwhile, since the raw material of the sugar solution of the invention of the present application is rice (non-glutinous rice), which is a natural product, and the sugar solution is composed of a decomposition product thereof, the sugar solution of the present invention contains a very wide variety of components in addition to sugar and soluble protein. Thus, it is considered that not only some of these components produce a good taste and flavor of the sugar solution, but also components act synergistically to provide such a taste and flavor. Specifying and/or quantifying the respective components responsible for the taste and flavor to identify the constitution of the sugar solution of the present invention is impossible, or very impractical, considering an enormous amount of time and money required to do so and detection limits of analytical equipment.

Thus, directly specifying the features of the invention of the present application by further configurations or characteristics of a matter is impossible or impractical.

### BRIEF DESCRIPTION OF DRAWINGS

FIG. 1 is a schematic view illustrating an example of methods for measuring volume of a sediment.

FIG. 2 is an outline of the method for producing a sugar solution of the present invention, schematically showing apparatus used for the production.

### DESCRIPTION OF EMBODIMENTS

The present invention will be described in more detail.

The present invention is a sugar solution comprising water and a reaction product of rice and α amylase in the water, wherein the amount of soluble protein in the sugar solution is 200 μg/ml or less and the amount of an extract component in the sugar solution is 43% by weight or more based on the total amount of the sugar solution.

A reaction for preparing the reaction product of rice and α amylase is a reaction performed at a temperature of 90° C. to 150° C. for 1 minute to 3.5 minutes, with blowing water vapor into a liquefied solution or an emulsified solution prepared from rice, α amylase and water, and

the time of liquefaction for preparing the liquefied solution or the emulsified solution from rice, α amylase and water is 15 seconds to 30 minutes and the temperature of liquefaction is 55° C. to 80° C.

The liquefied solution or emulsified solution used for producing the sugar solution of the present invention is not limited by requirements other than those specified above, as long as it can be used as a raw material for preparing the desired sugar solution. Furthermore, conditions other than the time of liquefaction and the temperature of liquefaction when performing liquefaction to obtain the liquefied solution or emulsified solution are not limited, and conditions may be those usually used in this technical field. The method for mixing water, rice and α amylase, which are raw materials of the liquefied solution or emulsified solution, is not limited. As used herein, “liquefaction” means decomposition of starch or sugar by an enzymatic reaction with a change in properties. In the case of starch, this refers to a phenomenon in which amylase acts on and decomposes starch which has been gelatinized by heating. As a result of liquefaction, chains in starch are roughly cut to short ones so that they are soluble in water. In the present description, the term “emulsification” and “emulsified product” may be alternatively used depending on the degree of liquefaction.

In the present invention, the sugar solution, which is a reaction product of rice and amylase, refers to an aqueous solution containing sugar formed when starch chains which have been cut short in liquefaction are cut even shorter by amylase (α amylase or glucoamylase, and preferably α amylase). In the sugar solution, most of the original starch is decomposed into glucose and some into oligosaccharide. The sugar solution of the present invention also includes a solution prepared by slightly concentrating the original sugar solution containing a reaction product of rice and amylase to have a desired composition of components and a solution prepared by further decomposing oligosaccharide in the reaction product to have a desired composition of components.

The type of α amylase used in the present invention is not limited, and α amylase such as α amylase 800 (made by HBI Enzymes Inc.), Kokulase G2 (made by Mitsubishi-Chemical Foods Corporation), Kleistase U50 (made by Daiwa Fine Chemicals Co., Ltd.) and Spitase CP (made by NAGASE & CO., LTD.) may be used.

The above concentrating includes concentrating to a volume of not less than about 90% of the volume of the sugar solution before concentration. It goes without saying that the amount of soluble protein does not exceed 200 μg/ml even if the sugar solution is concentrated.

The above further decomposition of oligosaccharide includes decomposition during being stored for about 20 minutes to about 22 hours at a temperature usually employed in decomposition of oligosaccharide by liquefying enzyme (e.g., α amylase) (about 55° C. to about 70° C.). The amount of the extract component and the amount of soluble protein do not substantially change even if the oligosaccharide is further decomposed as described above. The reason for no change in the amount of soluble protein even by further decomposition of the oligosaccharide is not theoretically restricted, and is considered to be because when rice, a raw material of the sugar solution of the present invention, is saccharified by α amylase at a temperature of 90° C. to 150° C. for 1 minute to 3.5 minutes, almost all soluble components are eluted and solubilized, and the remaining components are irreversibly modified (insolubilized), and thus no or few soluble components exist in the subsequent step of decomposition, and thus the amount of extract components and the amount of soluble protein do not increase. The original object of the above further decomposition is not to increase the amount of extract components by decomposition of raw material components, but is only to prevent softening, i.e., decay of a sugar solution with decomposition of oligosaccharide, occurring in storage before use for producing a seasoning or a food and a drink. Even if the further decomposition is performed at about 55° C. to about 70° C. for about 20 minutes to about 22 hours, these conditions are not for increasing the amount of extract components and soluble protein.

The soluble protein in the present invention means protein dissolved in water in a high temperature condition in the process for producing a sugar solution. Soluble protein is the main constituent of soluble components which are responsible for forming a sediment (precipitation) in unprocessed mirin or mirin.

The extract component in the present invention means soluble solids defined in the Japanese Liquor Tax Law, and is mainly composed of sugar and components such as soluble protein other than sugar. The main constituent of sugar in the extract component of a sugar solution used for mirin includes glucose and oligosaccharide.

In the sugar solution of the present invention, the amount of soluble protein is 200 μg/ml or less and the amount of the extract component in the sugar solution is 43% by weight or more based on the total amount of the sugar solution. The amount of soluble protein is relatively very smaller than the amount of sugar in the extract component compared with that in conventional sugar solutions prepared using rice. In the sugar solution of the present invention, the amount of the extract component is large, and thus the amount of sugar in the extract component is sufficiently large. In the sugar solution of the present invention, the ratio of sugar to the entire extract component is about 90% by weight or more, and in the case of a sugar solution for mirin, the ratio is about 92% by weight or more.

The amount of soluble protein in the sugar solution of the present invention is preferably 180 μg/ml or less, and more preferably 160 μg/ml or less. An amount of soluble protein in a sugar solution of about 400 μg/ml or less is usually practically acceptable.

A larger amount of an extract component is preferred in the sugar solution of the present invention, while the amount is preferably 43% by weight to 65% by weight, more preferably 45% by weight to 65% by weight based on the total amount of the sugar solution in consideration of production efficiency and manufacturing cost.

The method for measuring the amount of soluble protein in the sugar solution of the present invention is not limited as long as it is a usual method in this technical field. Examples thereof include measurement based on colorimetric analysis using a protein assay reagent made by Bio-Rad Laboratories, Inc.

The method for measuring the amount of extract components in the sugar solution of the present invention is not limited as long as it is a usual method in this technical field. Examples thereof include measurement methods based on the Official Analysis Method of the National Tax Agency. Quoting from the Official Analysis Method of the National Tax Agency, the method for measurement is as follows.

“A sample is diluted twice. The amount of extract components is calculated by the following formula, in which S represents the specific gravity measured according to 7-3 and A represents the specific gravity (15/15° C.) obtained by conversion as in Table 2 using ½ of the alcohol component measured according to 7-4.

E=[(S−A)×260+0.21]×2

(Note) Round off five decimal places in the calculation and round value E down to two decimal places.

The above “specific gravity measured according to 7-3” is measured by “A) hydrometer method” or “B) vibration densitometer method” (both methods are described in National Tax Agency's Instruction No. 1, Jan. 11, 1961, National Tax Agency's Instruction No. 6, 2007, and page 17, “The Official Analysis Method of the National Tax Agency,” published by the National Tax Agency).

Furthermore, the above “alcohol component measured according to 7-4” is measured by the method described in page 3 to page 8, “The Official Analysis Method of the National Tax Agency” described above.

The sugar solution of the present invention includes a solution prepared by slightly concentrating the original sugar solution containing a reaction product of rice and amylase to have a desired composition of components as described above. Of the sugar solutions of the present invention, a solution containing sugar itself in the reaction product of rice and amylase without the step of concentration is preferred because it is easy to produce the solution. However, of the sugar solutions of the present invention, a solution prepared by subjecting the original sugar solution containing a reaction product of rice and amylase to a step of concentration is preferred because the amount of components can be adjusted.

Furthermore, in the sugar solution of the present invention, the amount of sugar and/or soluble protein in the reaction product of rice and amylase may be slightly adjusted by adding saccharide and/or by diluting the reaction product itself. The addition of saccharide includes addition of sugar such as glucose, or oligosaccharide, and mixing with a sugar solution containing abundant extract components. The above dilution includes dilution to a volume not more than about 105% of the volume of the sugar solution before dilution with water or the like and mixing with a sugar solution containing extract components at a lower concentration.

When the sugar solution of the present invention is prepared with slightly adjusting the amount of sugar and/or soluble protein in the reaction product of rice and amylase by adding saccharide and/or by diluting the reaction product itself as described above, the reaction product itself having a value of (the amount of the extract component in the reaction product [% by weight]/the amount of soluble protein in the reaction product [w/v %]) of 2,000 or more is useful as a precursor for producing the sugar solution of the present invention (Present Invention [9]).

The above numerical range of 2,000 or more is determined by converting the amount of soluble protein (μg/ml) into w/v % with converting the weight of a sugar solution into grams and simply calculating the ratio to the value of the amount of an extract component (% by weight) (the amount of the extract component [% by weight])/(the amount of soluble protein [%]). The ratio is, for example, 2,250 (43/0.02=2,250) or more in the sugar solution of the present invention. By contrast, in conventional sugar solutions, the ratio is at most about 1,500.

The size and form of rice, which is a raw material for the sugar solution of the present invention, are not limited, and it may be grain rice, broken rice, rice flour or sake lees. The sugar solution of the present invention comprising rice flour as a raw material is preferred (Present Invention [3]). For the type of rice, the sugar solution of the present invention using non-glutinous rice is also preferred.

Rice for beer is also preferably used in the present invention. The rice for beer refers to a non-standard, broken rice whose size and color do not to meet, the specific standard (the standard of screening includes size (1.7 mm or 1.8 mm) and color (whiteness)) and fall far short of the size and the degree of whiteness.

Amylase used in the present invention includes α amylase, glucoamylase and protease, and high temperature resistant amylase is used as amylase without limitation.

For Application of Sugar Solutions [4] to [7] of the Present Invention

The sugar solution of the present invention is used for producing a seasoning or a food and a drink. Discoloration of the sugar solution of the present invention is much smaller than that of conventional sugar solutions, in particular, those concentrated or produced by heating long time. Thus, the sugar solution of the present invention does not reduce the value of products prepared by mixing the sugar solution due to unnecessary discoloration. Examples of seasonings include mirin, dressing, soup bases such as noodle soup base, hot pot soup base and broth soup, and sauces for barbeque, skewered chicken and fermented soy beans.

Examples of food and drink include (1) processed meat products such as ham, sausages, Hamburg steaks and meatballs, (2) processed fish products such as fish cakes, tube-shaped fish cakes, fish meatballs and fish sausages, (3) dairy products such as cheese, processed cheese products and yogurt, (4) drinks such as fruit juices, coffee drinks, tea drinks, milk, milk-based drinks, probiotic drinks, soy milk, sports drinks, energy drinks and soft drinks such as carbonated water, and alcohol drinks such as beer, sake (rice wine), plum wine, liqueur and sweet sake, (5) soups such as cream soup, clear soup and Chinese soup, (6) bread products such as pastries, baguette and white bread, (7) cereals such as cornflakes and brown rice flakes, (8) noodles such as soba noodle and udon noodle, (9) pastas such as spaghetti and macaroni, (10) powder-based food including powder mix such as Japanese savory pancake mix and steamed bun mix, (11) Western confectionary such as cookies and pies, (12) Japanese confectionary such as rice cake confections, kudzu starch noodles, glutinous rice flower and yokan (sweet bean jelly), and (13) desserts such as jellies using gelatin, agar or pectin.

The method for preparing a seasoning or a food and a drink using the sugar solution of the present invention is not limited. They may be produced by a usual method in this technical field, including the step of mixing the sugar solution of the present invention with other materials. For example, mirin may be produced by adding liquid sugar, alcohol such as shochu (Japanese spirits) and rice koji to the sugar solution of the present invention to prepare moromi (sake mash), and compressing the mixture and then pasteurizing it at a temperature of 115° C. or more, and removing sediment and filtering.

The sugar solution of the present invention is particularly preferably used for producing a seasoning, mirin. This is because since the amount of soluble protein based on the amount of extract components is significantly smaller than that in conventional sugar solutions, mirin can be produced efficiently at high yield (Present Inventions [5] and [6]).

The present invention also provides a sugar solution in which the volume of a sediment before removing the sediment is 35 or less, and the amount of soluble protein is 200 μg/ml or less and the amount of an extract component is 43% by weight or more based on the total amount (Present Invention [10]). The sugar solution is not specified by the condition of emulsification or digestion in production. This sugar solution is also preferred because it enables efficient production of mirin.

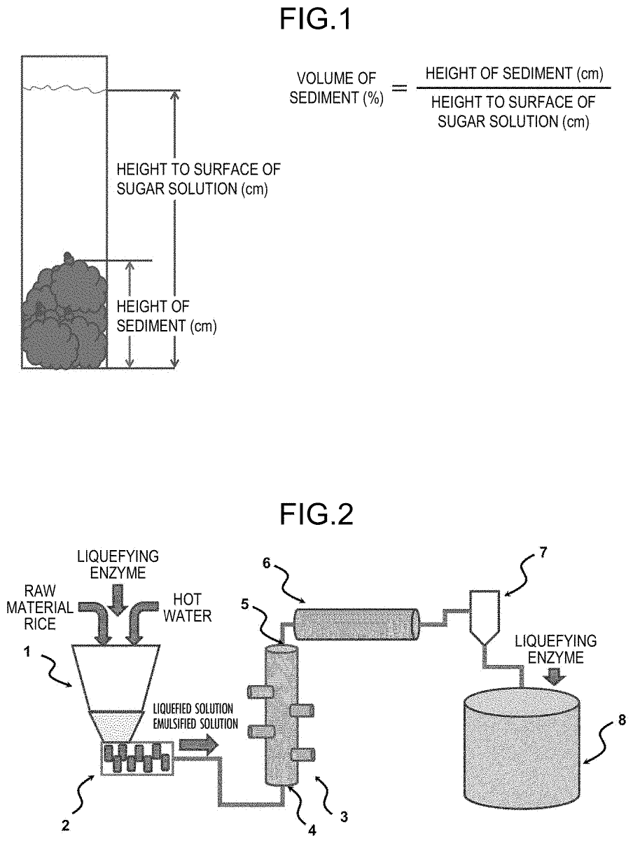

Of the sugar solutions of the present invention, those which provides, with a usual method, unprocessed mirin in which the value of the volume of a sediment (%) is 35 or less are preferred, those which provides unprocessed mirin in which the value of the volume of the sediment (%) is 30 or less is more preferred, and those which provides unprocessed mirin in which the value of the volume of the sediment (%) is 25 or less are further preferred. In this regard, the value of the volume of a sediment may be represented by the ratio of the height of the sediment to the height to the surface of a solution (%), the sediment being produced by pouring, into a container having a substantially uniform shape in a longitudinal direction, a sugar solution (e.g., unprocessed mirin) after pasteurization and before removing the sediment, together with an aggregate, carrageenan (for example, a 0.5% solution in an amount of 0.24 ml/100 ml) and optionally activated carbon (in an amount of, for example, 0.3 g/100 ml), and stirring the mixture and then allowing the mixture to stand indoors for 3 days (FIG. 1). The whole shape of the above container is not particularly limited, and a cylindrical glass container such as a graduated cylinder may be used. The size of the container is not limited, and the container may have an inner diameter of about 2.5 cm to 3.5 cm and a height of 18 cm to 23 cm. A container having an inner diameter of about 3.2 cm and a height of about 20 cm is preferably used. A cylindrical glass container having an inner diameter of 3.2 cm and a height of 20 cm is most preferred.

It is preferable that when a sugar solution is poured into a container having a substantially uniform shape in the longitudinal direction, the sugar solution is poured into the container to a depth of about 90% or more of the depth of the container.

When a cylindrical glass container having an inner diameter of 3.2 cm and a height of 20 cm is used, the value of the volume of a sediment in a conventional sugar solution is about 40, and in this case the amount of the corresponding soluble protein is about 200 μg/ml.

The present invention also relates to a seasoning or a food and a drink, prepared by using the sugar solution of the present invention (Present Invention [6]). Examples of seasonings prepared by using the sugar solution of the present invention include mirin, dressing, soup bases such as noodle soup base, hot pot soup base and broth soup, and sauces for barbeque, skewered chicken, fermented soy beans and the like.

Examples of food and drink prepared by using the sugar solution of the present invention include (1) processed meat products such as ham, sausages, Hamburg steaks and meatballs, (2) processed fish products such as fish cakes, tube-shaped fish cakes, fish meatballs and fish sausages, (3) dairy products such as cheese, processed cheese products and yogurt, (4) drinks such as fruit juices, coffee drinks, tea drinks, milk, milk-based drinks, probiotic drinks, soy milk, sports drinks, energy drinks and soft drinks such as carbonated water, and alcohol drinks such as beer, sake (rice wine), plum wine and liqueur, (5) soups such as cream soup, clear soup and Chinese soup, (6) bread products such as pastries, baguette and white bread, (6) cereals such as cornflakes and brown rice flakes, (8) noodles such as soba noodle and udon noodle, (9) pastas such as spaghetti and macaroni, (10) powder-based food including powder mix such as Japanese savory pancake mix and steamed bun mix, (11) Western confectionary such as cookies and pies, (12) Japanese confectionary such as rice cake confections, kudzu starch noodles, glutinous rice flower and yokan (sweet bean jelly), and (13) desserts such as jellies using gelatin, agar or pectin.

Of the seasonings or food and drink prepared by using the sugar solution of the present invention, mirin is preferred (Present Inventions [5] and [7]).

The method for producing the sugar solution of the present invention is not limited. The sugar solution is produced, for example, by a method comprising performing a reaction at about 90° C. to about 150° C. for about 1 minute to about 3.5 minutes with blowing water vapor into a liquefied solution or an emulsified solution prepared from rice, amylase and water (a slurry product prepared from amylase and water and having a relatively high viscosity) to give a reaction product as a sugar solution, in which the amount of soluble protein is 200 μg/ml or less and the amount of an extract component is 43% by weight or more based on the total amount.

Other conditions of liquefaction for preparing the above liquefied solution or emulsified solution are not limited as long as the conditions are those usually substantially used in this technical field.

A sugar solution is preferred, which is prepared by further concentrating and/or storing the sugar solution, i.e., the above reaction product, and in which the amount of soluble protein is 200 μg/ml or less and the amount of an extract component is 43% by weight or more based on the total amount of the sugar solution, wherein the concentrating is a concentrating to a volume of not less than 90% of the volume of the sugar solution before concentration and the storing is storing at 55° C. to 70° C. for 20 minutes to 22 hours with decomposition of oligosaccharide in the reaction product by a liquefying enzyme containing α amylase. This sugar solution is preferred because it can be stored until use for producing a seasoning or a food and a drink (Present Invention [2]). The liquefying enzyme may or may not be added in the storage. When a liquefying enzyme is added, an enzyme other than α amylase may be added alone or in combination with α amylase as a liquefying enzyme. Examples of enzymes other than α amylase include, but are not limited to, protease, glucoamylase and cellulase. The total amount of the above liquefying enzymes added (including α amylase additionally added) is not limited, and may be about 1/1,000 or less of the weight of rice added.

When no liquefying enzyme is added in the storage, the decomposition of reaction product by α amylase, which is coexistent with the reaction product and used for saccharification, may slightly take place.

Of the sugar solutions of the present invention, a sugar solution is preferred, wherein a reaction for preparing the reaction product of rice and amylase is a reaction performed at a temperature of about 100° C. to about 150° C. for about 1 minute to 2 minutes, or about 90° C. to about 135° C. for more than 2 minutes and about 3.5 minutes, with blowing water vapor into a liquefied solution or an emulsified solution prepared from rice, amylase and water.

The above temperature may be changed depending on the reaction time, and may be relatively low when the reaction time is long. For example, it is preferable that when the reaction time is about 1 minute to 2 minutes, the temperature is 110° C. or more. It is preferable that when the reaction time is about 2 minutes to 3 minutes, the temperature is relatively low, for example, 130° C. or less. It is preferable that in the reaction for preparing the reaction product of rice and amylase, the whole mixture of rice, amylase and water is in homogenous reaction conditions. It is preferable that shear force is applied to the mixture of rice, amylase and water, the mixture is fed, and/or the mixture is stirred when the reaction is performed.

Present Invention [8] (Method for Producing Sugar Solution)

The present invention also relates to the following method for producing a sugar solution:

a method for producing a sugar solution, comprising performing a reaction at a temperature of about 90° C. to about 150° C. for about 1 minute to about 3.5 minutes, with blowing water vapor into a liquefied solution or an emulsified solution prepared from rice, amylase and water to give a reaction product as a sugar solution, in which the amount of soluble protein is 200 μg/ml or less and the amount of an extract component is 43% by weight or more based on the total amount. In this regard, the time of liquefaction for preparing the liquefied solution or the emulsified solution from rice, amylase and water is 15 seconds to 30 minutes and the temperature of liquefaction is 55° C. to 80° C.

The temperature of liquefaction needs not to be constant and may vary during liquefaction in the range of about 55° C. to about 80° C., for the whole mixture of rice, amylase and water to be liquefied, or in some part thereof. It is preferable that the mixture is liquefied with stirring during liquefaction. The speed and frequency of stirring are not limited.

The above production method provides not only the sugar solution of the present invention but also a sugar solution which can be a raw material for the sugar solution of the present invention.

The reason why the production method of the present invention provides the sugar solution of the present invention is not theoretically restricted, and seems to be because components of rice are decomposed in a short time and thus the degree of heat denaturation of protein is much smaller than that in conventional methods, and consequently production of soluble (water-soluble) protein is also significantly suppressed. While enzymatic reaction occurs for about 1 minute to 3.5 minutes in the above production method of the present invention, enzymatic reaction (digestion) in conventional methods takes about 2 hours, which is much longer than that in the method of the present invention. This is because, in conventional methods, a raw material containing rice and amylase is introduced into hot water in a digestion tank while stirring and circulating the hot water, and the digestion tank is continued to be heated to increase the temperature to about 80° C. even after the introduction.

Although methods designed for increasing extract components have been reported in conventional art, suppression of formation of water-soluble protein has not been even studied. The major significance of the present invention is the adoption of a combination of a temperature and a time most suitable for performing an enzymatic reaction in an extremely short time.

Of the methods of the present invention, a method is preferred, in which a reaction for preparing the reaction product of rice and amylase is a reaction performed at a temperature of about 100° C. to about 150° C. for about 1 minute to 2 minutes, or about 90° C. to about 135° C. for more than 2 minutes and about 3.5 minutes, with blowing water vapor into a liquefied solution or an emulsified solution prepared from rice, amylase and water.

The above temperature may be changed depending on the reaction time, and may be relatively low when the reaction time is long. For example, it is preferable that when the reaction time is about 1 minute to about 2 minutes, the temperature is 110° C. or more. It is preferable that when the reaction time is about 2 minutes to about 3 minutes, the reaction temperature is relatively low, for example, 130° C. or less. It is particularly preferable that the reaction time is about 1.5 minutes and the temperature is 110° C. to 120° C.

Referring to the equipment used for the method of the present invention, the reactor for saccharification is not limited, as long as it is capable of performing enzymatic reaction of a high viscosity material (about 450 cps or more) at the desired temperature for the desired time. Examples of such equipment include RotaTherm (registered trademark), KID's Cooker (registered trademark) and what is called a jet cooker.

RotaTherm is preferred because it can feed, by pumping, even materials which cannot be fed by pumping in conventional methods due to a sudden increase in the viscosity during liquefication, based on a suitable effect of shearing. When the amount of materials is relatively small, KID's Cooker is also preferably used.

Of the production methods of the present invention, a method is preferred, including a treatment of instantly heating a liquefied solution or an emulsified solution and rapidly gelatinizing and mixing the material by direct heating such as application of steam jet or indirect heating from the outside, thereby decomposing starch in the liquefied solution or the emulsified solution. Any of RotaTherm, KID's Cooker and the jet cooker described above may be used as equipment for performing such treatment.

It is preferable that the production method of the present invention includes heating a high viscosity liquefied solution or emulsified solution uniformly and preventing the liquefied solution or emulsified solution from being burnt, using, for example, cooling water.

Anon-limiting example of the production method of the present invention is schematically shown in FIG. 2. More specifically, first raw material rice, liquefying enzyme (e.g., α amylase) and hot water (about 50° C. to about 80° C.) are introduced into a mixing tank 1 to perform liquefaction in a temperature range of about 55° C. to about 80° C. to give a liquefied solution or an emulsified solution. The temperature of the above hot water introduced is suitably adjusted and maintained at the desired temperature of about 55° C. to about 80° C. Other conditions of liquefaction for preparing the above liquefied solution or emulsified solution are not limited as long as the conditions are those usually substantially used in this technical field. The above other conditions include:
The mixing ratio of hot water to rice (weight ratio) is about 90 to about 125 of hot water to 100 of rice.
The amount of liquefying enzyme to be used is about 1/5,500 to about 1/2,000 based on the weight of rice.
The time of liquefaction is about 15 seconds to about 30 minutes.

The resulting liquefied product in the form of slurry, i.e., a liquefied solution or an emulsified solution has a viscosity of about 450 to about 100,000 cps.

RotaTherm is preferred because it can feed, by pumping, even materials which cannot be fed by pumping in conventional methods due to a sudden increase in the viscosity during liquefication, based on a suitable effect of shearing. When the amount of materials is relatively small, KID's Cooker is also preferably used.

The time of liquefaction is inversely proportional to the viscosity of the product, i.e., the liquefied solution or emulsified solution. The advantage is that when the time of liquefaction is relatively short (e.g., 15 seconds to 20 seconds), the production efficiency is relatively high. Another advantage is that when the time of liquefaction is relatively long (e.g., about 10 minutes to about 30 minutes), a mixture having a relatively low viscosity is obtained and thus handleability in production is relatively good. It is preferable to change the time of liquefaction based on the equipment used and the schedule of production when practicing the production method of the present invention.

The above time of liquefaction for preparing the above liquefied solution or emulsified solution is preferably about 15 seconds to about 20 minutes, and more preferably about 15 seconds to about 10 minutes.

The temperature of liquefaction for preparing the above liquefied solution or emulsified solution is preferably about 55° C. to about 70° C., and more preferably about 55° C. to about 65° C. as described above. Furthermore, the temperature of liquefaction needs not to be constant and may vary during liquefaction in the range of about 55° C. to about 80° C., for the whole mixture of rice, amylase and water to be liquefied, or in some part thereof as described above. It is preferable that the mixture is liquefied with stirring, as described above. The mixing ratio of hot water to rice (weight ratio) in liquefaction for preparing the above liquefied solution or emulsified solution is preferably about 90 to about 125 of hot water based on 100 of rice, more preferably about 103 to about 119 of hot water based on 100 of rice. The amount of water is inversely proportional to the viscosity of the resulting liquefied solution or emulsified solution. Thus, it is also preferable to change the amount of water based on the equipment used and the schedule of production when practicing the production method of the present invention.

The amount of the above liquefying enzyme (amylase) to be used in liquefaction for preparing the above liquefied solution or emulsified solution is preferably about 1/10,000 to about 1/2,000, more preferably about 1/3,300 to about 1/2,000, and further preferably about 1/3,300 to about 1/3,000 based on the weight of rice. The amount of amylase is also inversely proportional to the viscosity of the resulting liquefied solution or emulsified solution. Thus, it is also preferable to change the amount of amylase based on the equipment used and the schedule of production when practicing the production method of the present invention.

Next, liquefaction of the liquefied solution or emulsified solution is performed (FIG. 2). In the following, an example using an emulsified solution will be described in detail.

The above emulsified solution produced is introduced into a reactor for saccharification 3 from the loading port of a transfer-type mill 2. High temperature water vapor (about 170° C.) is constantly blown into the reactor for saccharification 3 from the outside at some parts so that the water vapor is uniformly distributed in the reactor for saccharification 3 to heat the emulsified solution to a predetermined temperature. The amount and the rate of water vapor to be blown thereinto are not limited, and may be determined in consideration of the amount and the viscosity of the emulsified solution, the reaction temperature and the rate of feeding of the emulsified solution. The emulsified solution is transferred to the side of the discharge port 5 from the loading port 4 of the reactor 3 with stirring to perform liquefaction to give a sugar solution. The temperature in the reactor 3 is kept at a predetermined temperature in the range of about 90° C. to about 150° C. during transfer, and then the emulsified solution is sent to a holding pipe 6 through the discharge port 5. It is preferable that the enzymatic reaction is continuously performed in the holding pipe 6 for a predetermined time of about 1 minute to about 3.5 minutes with keeping the above temperature.

It is preferable that the material which has been passed through the holding pipe 6 is introduced into a buffer tank 8 to be exposed to an environment containing a liquefying enzyme (e.g., α amylase) so that the material is softened, or in other words, oligosaccharide is decomposed, if necessary. The retention time in the buffer tank 8 may be about 20 minutes to about 22 hours and the retention temperature may be about 55° C. to about 70° C. (hereinafter “retention” may be referred to as “holding”). The liquefying enzyme includes α amylase. This α amylase may be only contained in the material passed through the holding pipe 6, which is a reaction product resulting from the reaction between rice and α amylase performed by blowing water vapor into a liquefied solution or an emulsified solution at a temperature of 90° C. to 150° C. for 1 minute to 3.5 minutes (hereinafter the reaction may be referred to as “digestion” in the following description to mean the same reaction). Alternatively, α amylase may be additionally added thereto.

An enzyme other than α amylase may be added alone or in combination with a amylase as a liquefying enzyme. Examples of enzymes other than α amylase include, but are not limited to, protease, glucoamylase and cellulase. The total amount of the above liquefying enzyme added (including α amylase additionally added) is not limited, and may be about 1/10,000 to about 1/1,000 of the weight of rice added.

When α amylase, protease and glucoamylase are added to be used in combination as the liquefying enzyme, the amount of the respective liquefying enzymes may be about 1/30,000 to about 1/1,000 of the weight of rice added. Furthermore, the reaction product discharged from the discharge port 5 may be concentrated if necessary in an evaporation tank 7 before or after softening in the buffer tank 8. FIG. 2 shows an embodiment in which the reaction product is concentrated in the evaporation tank 7 before softening in the buffer tank 8, but the evaporation tank 7 may also be at the position where the reaction product which has been softened in the buffer tank 8 is concentrated. Volatile components contained in the reaction product which has been passed through the holding pipe 6 or the buffer tank 8 are removed in the evaporation tank 7.

The sugar solution produced by the production method of the present invention may be used for producing a seasoning or a food and a drink, and particularly preferably used for producing mirin.

For the production method for producing mirin using the sugar solution of the present invention, usual steps in this technical field are used except for using the sugar solution of the present invention as a sugar solution.

The present invention will be described in more detail with reference to Examples, but the scope of the present invention is not limited thereto in any respect.

### EXAMPLES

### <Example A> Production of Sugar Solution of Present Invention (Examples 1 to 5)

(Materials and methods) A non-glutinous rice flour was used as a raw material. For the type of rice flour, a rice flour prepared by finely pulverizing white rice was used.

A raw material rice, liquefying enzyme (α amylase: α amylase 800 (made by HBI Enzymes Inc.) and hot water of about 60° C. were introduced into a mixing tank, and the mixture was kept at 55° C. to perform liquefaction. The mixing ratio of hot water to rice (volume ratio) was 90 to 125 of hot water to 100 of rice. The amount of the liquefying enzyme added was 1/5,000 to 1/2,000 based on the weight of rice.

The emulsification (liquefaction) was performed for 30 minutes. The viscosity of the resulting emulsified solution (slurry) was about 500 cps to 100,000 cps.

The above emulsified solution produced was introduced into a reactor for saccharification (RotaTherm made by GoldPeg) through the loading port to carry out saccharification. In the reaction, water vapor at 110° C. was constantly blown into the reactor from the outside at some parts so that the water vapor was uniformly distributed in the reactor, and the emulsified solution was transferred to the discharge port from the loading port of the reactor with stirring. The temperature of the reactor was kept at the above predetermined temperature during the transfer.

The reaction time (digestion time) was 1.5 minutes.

After completion of the saccharification, the product (reaction product) discharged from the discharge port was transferred to the evaporation tank, where water corresponding to about 10% of the water vapor introduced was distilled off to concentrate the product. The concentration was performed in such a condition that the temperature of the solution treated was decreased to about 90° C. to about 70° C.

After the solution was cooled as described above, protease (Sumizyme LP made by Shin Nihon Chemical Co., Ltd.) was added thereto in an amount of 1/5,500 of the weight of the rice flour added, and the solution was held for about 18 hours in the holding tank while maintaining the temperature at 60° C.

(Evaluation) The amount of extract components (% by weight) was measured and calculated by the Official Analysis Method of the National Tax Agency. The following formula was used for calculation:

E (Amount of extract component (% by weight))=[(S−A)×260+0.21]×2

In the above formula, S represents the specific gravity of a specimen prepared by diluting the original sample to ½, which was determined by placing 100 ml of the specimen in a 100 ml graduated cylinder and measuring the weight. A represents the specific gravity (15/15° C.) obtained by conversion using ½ of the alcohol component measured according to the standard.

All Reference Examples and Comparative Examples did not contain an alcohol component. Thus, the amount of extract components was calculated with A=1.000 for convenience.

The amount of soluble protein (μg/mL) was measured by colorimetric analysis using a protein assay reagent made by Bio-Rad Laboratories, Inc.

(Results) The results are shown in Table 1.

As shown in the table, the sugar solution of the present invention contains a large amount of extract components (43.0% by weight to 53.3% by weight) and a small amount of soluble protein (37.4 μg/mL to 140.3 μg/mL).

TABLE 1

| | Temperature of | Time of | Time of | Temperature of | | | Amount of | Amount of |
|---|---|---|---|---|---|---|---|---|
| Example | liquefaction | liquefaction | digestion | digestion | Rice:hot | α | extract component | soluble protein |
| No. | [° C.] | [minutes] | [minutes] | [° C.] | water | amylase | [% by weight] | [μg/ml] |
| 1 | 55 | 30 | 1.5 | 100 | 1:1.19 | 1/3300 | 45.0 | 140.3 |
| 2 | | | | 110 | 1:1.19 | 1/3300 | 44.3 | 76.7 |
| 3 | | | | 110 | 1:1.25 | 1/3300 | 43.0 | 130.3 |
| 4 | | | | 110 | 1:1.03 | 1/2000 | 52.5 | 127.4 |
| 5 | | | | 110 | 1:0.9 | 1/3300 | 53.3 | 96.5 |

In Example 1 and Example 2, the volume of a sediment of unprocessed mirin, which was added to prepare mirin, was measured. As a result, the volume of the sediment was 21.9% and 18.3%, respectively. For the measurement of the volume of the sediment, the ratio of the height of the sediment to the height to the surface of the unprocessed mirin (%) was calculated, the sediment being produced by pouring the unprocessed mirin (after pasteurization and before removing the sediment) into a glass cylinder (inner diameter 3.2 cm, height 20 cm) together with activated carbon (0.3 g/100 ml) and an aggregate, carrageenan (a 0.5% solution in an amount of 0.24 ml/100 ml), and stirring the mixture and then allowing the mixture to stand indoors for 3 days, as described above.

### <Example B> Modification of Temperature of Liquefaction

(Materials and methods) A non-glutinous rice flour was used as a raw material.

A raw material rice, liquefying enzyme (α amylase) and hot water were introduced into a mixing tank, and the mixture was kept at 55° C., 65° C. or 80° C. to perform liquefaction. The mixing ratio of hot water to rice (volume ratio) was 119 of hot water to 100 of rice. The amount of the liquefying enzyme added was 1/5,000 to 1/3,300 based on the weight of rice.

The emulsification (liquefaction) was performed for 30 minutes. The viscosity of the resulting emulsified solution (slurry) was about 500 cps.

The above emulsified solution produced was introduced into a reactor for saccharification (RotaTherm made by GoldPeg) through the loading port to carry out saccharification. In the reaction, water vapor at 110° C. was constantly blown into the reactor from the outside at some parts so that the water vapor was uniformly distributed in the reactor, and the emulsified solution was transferred to the discharge port from the loading port of the reactor with stirring. The temperature of the reactor was kept at the above predetermined temperature during the transfer.

The reaction time (digestion time) was 1.5 minutes.

After completion of the saccharification, the product (reaction product) discharged from the discharge port was transferred to the evaporation tank, where the product was concentrated to distill off water corresponding to about 10% of the water vapor introduced. The concentration was performed in such a condition that the temperature of the solution treated was decreased to about 90° C. to about 70° C.

After the solution was cooled as described above, protease (Sumizyme LP made by Shin Nihon Chemical Co., Ltd.) was added thereto in an amount of 1/5,500 of the weight of the rice flour added (Example 8), or it was not added thereto (Examples 6 and 7), and the solution was held for about 18 hours in the holding tank while maintaining the temperature at 60° C.

(Evaluation) The resultant was evaluated in the same manner as in Example A.

(Results) The results are shown in Table 2.

As shown in the table, the sugar solution of the present invention contains a large amount of extract components (44.7% by weight to 46.8% by weight) and a small amount of soluble protein (3.1 μg/mL to 166 μg/mL).

The above results show that for the sugar solution of the present invention, the temperature in liquefaction may be 55° C. to 80° C.

TABLE 2

| | Temperature of | Time of | Time of | Temperature of | | | Amount of | Amount of |
|---|---|---|---|---|---|---|---|---|
| Example | liquefaction | liquefaction | digestion | digestion | Rice:hot | α | extract component | soluble protein |
| No. | [° C.] | [minutes] | [minutes] | [° C.] | water | amylase | [% by weight] | [μg/ml] |
| 6 | 55 | 30 | 1.5 | 110 | 1:1.19 | 1/3300 | 45.8 | 54.6 |
| 7 | 65 | | | | | 1/5000 | 46.8 | 166 |
| 8 | 80 | | | | | 1/5000 | 44.7 | 3.1 |

### <Example C> Verification of No Influence of Retention

(Materials and methods) A non-glutinous rice flour was used as a raw material. Rice flour generated in the process of polishing rice suitable for sake making was used.

A raw material rice, liquefying enzyme (α amylase: α amylase 800 (made by HBI Enzymes Inc.) and hot water were introduced into a mixing tank, and the mixture was kept at 55° C. to perform liquefaction. The mixing ratio of hot water to rice (volume ratio) was 119 of hot water to 100 of rice. The amount of the liquefying enzyme added was 1/5,000 based on the weight of rice.

The emulsification (liquefaction) was performed for 30 minutes. The viscosity of the resulting emulsified solution (slurry) was about 500 cps.

The above emulsified solution produced was introduced into a reactor for saccharification (RotaTherm made by GoldPeg) through the loading port to carry out saccharification. In the reaction, water vapor at 110° C. was constantly blown into the reactor from the outside at some parts so that the water vapor was uniformly distributed in the reactor, and the emulsified solution was transferred to the discharge port from the loading port of the reactor with stirring. The temperature of the reactor was kept at the above predetermined temperature during the transfer.

The reaction time (digestion time) was 1.5 minutes.

After completion of the saccharification, the product (reaction product) discharged from the discharge port was transferred to the evaporation tank, where water corresponding to about 10% of the water vapor introduced was distilled off to concentrate the product. The concentration was performed in such a condition that the temperature of the solution treated was decreased to about 90° C. to about 70° C.

After being cooled as described above, the solution was held for about 18 hours in the holding tank while maintaining the temperature at 60° C. without adding a liquefying enzyme.

(Evaluation) The resultant was evaluated in the same manner as in Example A. Samples were collected both before and after the storage for about 18 hours.

(Results) The results are shown in Table 3.

As shown in the table, it was confirmed that the sugar solution of the present invention contains the desired amount of extract components and the desired amount of soluble protein even after being stored for 18 hours.

TABLE 3

| | | | | | | | Amount of | | Amount of | |
|---|---|---|---|---|---|---|---|---|---|---|
| | | | | | | | extract component | | soluble protein | |
| | Temperature of | Time of | Time of | Temperature of | | | [% by weight] | | [μg/ml] | |
| Example | liquefaction | liquefaction | digestion | digestion | Rice:hot | α | Before | After | Before | After |
| No. | [° C.] | [minutes] | [minutes] | [° C.] | water | amylase | holding | holding | holding | holding |
| 9 | 55 | 30 | 1.5 | 110 | 1:1.19 | 1/5000 | 43.4 | 43.0 | 36.4 | 37.4 |
| 10 | | | | | | | 52.4 | 52.5 | 53.1 | 73.1 |

### <Example D> Consideration of Time of Liquefaction and Time of Retention

(Materials and Methods)

A non-glutinous rice flour was used as a raw material.

A raw material rice, liquefying enzyme (α amylase: α amylase 800 (made by HBI Enzymes Inc.) and hot water of 70° C. were introduced into a mixing tank, and mixed. The mixing ratio of hot water to rice (volume ratio) was 100 of hot water to 100 of rice. The amount of the liquefying enzyme added was 1/2,000 based on the weight of rice.

The time from mixing to heating was 15 seconds, 10 minutes or 20 minutes. The viscosity of the resulting emulsified solution (slurry) was about 100,000 cps.

The above emulsified solution produced was introduced into a reactor for saccharification (RotaTherm made by GoldPeg) through the loading port to carry out saccharification. In the reaction, water vapor at 110° C. was constantly blown into the reactor from the outside at some parts so that the water vapor was uniformly distributed in the reactor, and the emulsified solution was transferred to the discharge port from the loading port of the reactor with stirring. The temperature of the reactor was kept at the above predetermined temperature during the transfer.

The reaction time (digestion time) was 1.5 minutes.

After completion of the saccharification, the product (reaction product) discharged from the discharge port was transferred to the evaporation tank, where water corresponding to about 10% of the water vapor introduced was distilled off to concentrate the product. The concentration was performed in such a condition that the temperature of the solution treated was decreased to about 110° C. to about 70° C.

After the solution was cooled as described above, α amylase was added thereto in an amount of 1/10,000, glucoamylase in an amount of 1/10,000, and protease in an amount of 1/10,000 of the weight of the rice flour, and the solution was held for about 16 hours or about 22 hours in the holding tank while maintaining the temperature at 65° C.

(Evaluation) The resultant was evaluated in the same manner as in Example A.

(Results) The results are shown in Table 4.

The results show that the sugar solution of the present invention can be produced even when the time of liquefaction is less than 30 minutes.

TABLE 4

| | Temperature of | Time of | Time of | Temperature of | | Time of | Amount of | Amount of |
|---|---|---|---|---|---|---|---|---|
| Example | liquefaction | liquefaction | digestion | digestion | Rice:hot | retention | extract component | soluble protein |
| No. | [° C.] | [minutes] | [minutes] | [° C.] | water | [hours] | [% by weight] | [μg/ml] |
| 11 | 60 | 15 seconds | 1.5 | 110 | 1:1 | 22 | 50.3 | 55.2 |
| 12 | | 10 minutes | | | | 16 | 50.2 | 147.2 |
| 13 | | 20 minutes | | | | 16 | 50.2 | 159.2 |

### <Example E> Changing Equipment of Production: Production of Sugar Solution Using KID's Cooker (Apparatus for Heating and Sterilizing Liquid)

(Materials and methods) A sugar solution was produced in the same manner as in Examples above except for using KID's Cooker (made by Okawara Mfg. Co., Ltd.) as a reactor for saccharifying the liquefied solution (emulsified solution) prepared.

(Evaluation) The amount of extract components (% by weight) was measured by the Official Analysis Method of the National Tax Agency.

(Results) The results are shown in Table 5. As shown in the table, a sugar solution containing a large amount of extract components was produced by the production method of the present invention using KID's Cooker.

TABLE 5

| | Temperature of | Time of | Time of | Temperature of | | | Amount of | Amount of |
|---|---|---|---|---|---|---|---|---|
| Example | liquefaction | liquefaction | digestion | digestion | Rice:hot | α | extract component | soluble protein |
| No. | [° C.] | [minutes] | [minutes] | [° C.] | water | amylase | [% by weight] | [μg/ml] |
| 14 | 60 | 10 | 1.5 | 110 | 1:1.10 | 1/3300 | 45.4 | 17.1 |

### <Example F> Production and Evaluation of Seasoning of Present Invention (Mirin)

(Materials and methods) Mirin was produced by a usual method using the sugar solution shown in Example 1 and Example 2.

(Evaluation) The chromaticity of the mirin produced as described above was measured by using a spectrophotometer (made by Shimadzu Corporation, UV-1600) (OD 430 nm).

(Results) The chromaticity of the mirin produced by using the sugar solution of Example 1 and the sugar solution of Example 2 was 0.163 and 0.158, respectively, which is significantly smaller than the chromaticity of conventional mirin (about 0.316) in which the sugar solution of the present invention is not used.

This shows that unnecessary discoloration is not caused and thus the value of products is not reduced in products such as seasonings produced by using the sugar solution of the present invention. The above seems to be because the discoloration of the sugar solution of the present invention is much smaller than that of conventional sugar solutions.

### Example A-1

(Materials and methods) A non-glutinous rice flour was used as a raw material.

A raw material rice, liquefying enzyme (α amylase: α amylase 800 (made by HBI Enzymes Inc.) and hot water were introduced into a mixing tank, and the mixture was heated to 80° C. to perform liquefaction. The mixing ratio of hot water to rice (volume ratio) was 110 of hot water to 100 of rice. The amount of the liquefying enzyme added was 1/3,300 based on the weight of rice.

The emulsification was performed for 10 minutes. The viscosity of the resulting emulsified solution (slurry) was about 500 cps.

The above emulsified solution produced was introduced into a reactor for saccharification (RotaTherm made by GoldPeg) through the loading port to carry out saccharification. In the reaction, water vapor at 110° C. was constantly blown into the reactor from the outside at multiple points so that the water vapor was uniformly distributed in the reactor, and the emulsified solution was transferred to the discharge port from the loading port of the reactor with stirring. The temperature of the reactor was kept at the above predetermined temperature during the transfer.

The reaction time (digestion time) was 1.5 minutes.

After completion of saccharification, the product (reaction product) discharged from the discharge port was transferred to the evaporation tank, where water corresponding to about 10% of the water vapor introduced was distilled off to concentrate the product. The concentration was performed in such a condition that the temperature of the solution treated was decreased to about 90° C. to about.

(Evaluation) The amount of extract components (% by weight) was measured and calculated by the Official Analysis Method of the National Tax Agency. The following formula was used for calculation:

E (Amount of extract component (% by weight))=[(S−A)×260+0.21]×2

In the above formula, S represents the specific gravity of a specimen prepared by diluting the original sample to ½, which was determined by placing 100 ml of the specimen in a 100 ml graduated cylinder and measuring the weight. A represents the specific gravity (15/15° C.) obtained by conversion using ½ of the alcohol component measured according to the standard.

All Examples and Comparative Examples did not contain an alcohol component. Thus, the amount of extract components was calculated with A=1.000 for convenience.

The amount of soluble protein (μg/mL) was measured by colorimetric analysis using a protein assay reagent made by Bio-Rad Laboratories, Inc.

Furthermore, the chromaticity was measured by using a spectrophotometer (made by Shimadzu Corporation, UV-1600) (OD 430 nm).

### Examples A-2 to A-4

A sugar solution was produced in the same manner as in Example A-1 except for changing the temperature of digestion to 120° C., 130° C. and 140° C., respectively. The amount of extract components [% by weight], the amount of soluble protein [μg/mL] and the chromaticity [OD430 nm] were also measured in the same manner as in Example A-1.

### Examples B-1 to B-4

The sugar solutions of Examples B-1 to B-4 were produced in the same manner as in A-1 to A-4 except for changing the time of digestion to 3.0 minutes. The amount of extract components [% by weight], the amount of soluble protein [μg/mL] and the chromaticity [OD430 nm] were also measured in the same manner as in Examples A-1 to A-4.

### COMPARATIVE EXAMPLES

The sugar solution of Comparative Example 1 was produced in the same manner as in Example A-1 except for changing the temperature of digestion to 95° C. Furthermore, the sugar solution of Comparative Example 2 was produced in the same manner as in Example A-4 except for changing the time of digestion to 3.0 minutes.

A sugar solution was prepared by using glutinous rice in Comparative Example 3 and a sugar solution was prepared by using non-glutinous rice in Comparative Example 4.

(2) Seasoning (Unprocessed Mirin)

Unprocessed mirin was produced by using the respective sugar solutions of Examples and Comparative Examples 1 to 3, and generation of a sediment was investigated by measuring the volume of the sediment in the respective cases.

For the measurement of the volume of the sediment, the ratio of the height of the sediment to the height to the surface of the unprocessed mirin (%) was calculated, the sediment being produced by pouring the unprocessed mirin (after pasteurization and before removing the sediment) into a glass cylinder (inner diameter 3.2 cm, height 20 cm) together with activated carbon (0.3 g/100 ml) and an aggregate, carrageenan (a 0.5% solution in an amount of 0.24 ml/ml), and stirring the mixture and then allowing the mixture to stand indoors for 3 days.

(Results) The results are shown in Table 6.

As shown in the table, the sugar solutions of the present invention (Examples A-1 to A-4 and B-1 to B-4) contains a large amount of an extract component (46.8% by weight to 53.0% by weight) and a small amount of soluble protein (108.0 μg/mL to 141.3 μg/mL). In particular, the sugar solutions of Examples A-1 to A-4 in which the time of digestion was short (1.5 minutes), in which the amount of extract components is small, are more preferred.

By contrast, the volume of the sediment of the unprocessed mirin of Comparative Example 1 was much larger than the volume of the sediment of the unprocessed mirin of Example B-1 in which the amount of soluble protein was slightly less than 200 μg/mL. Thus, the amount of soluble protein is estimated to be higher than 200 μg/mL in Comparative Example 1.

The amount of sugar in the extract component was small in the sugar solution of Comparative Example 2. The amount of soluble protein was large in Comparative Example 3. The sugar solution of Comparative Example 2, however, may be used as a material for the sugar solution of the present invention, because the ratio of the amount of extract components to the amount of soluble protein is large ((A)/(B)×10,000=2,715), although the amount of extract components themselves is 42.0% by weight, which is less than 43% by weight.

The sugar solution of Comparative Example 4 had high chromaticity (0.162) with larger discoloration than that of the sugar solution of the present invention. Thus, the amount of soluble protein is assumed to be larger than that of the sugar solution of the present invention.

TABLE 6

| | | | | Amount of | Amount of | | Volume of | |
|---|---|---|---|---|---|---|---|---|
| | | Time of | Temperature of | extract component | soluble protein | | sediment of | |
| | | digestion | digestion | [% by weight] | [μg/ml] | Chromaticity | unprocessed | (A)/(B) × |
| Category | No. | [minutes] | [° C.] | (A) | (B) | [OD430 nm] | mirin [%] | 10000 |
| Comparative | 1 | 1.5 | 95 | 54.6 | >200 | 0.072 | 48.4 | <2730 |
| Example | | | | | | | | |
| Example | A-1 | | 110 | 52.6 | 121.3 | 0.068 | 20 | 4336 |
| | A-2 | | 120 | 53.0 | 120.0 | 0.072 | 4.6 | 4417 |
| | A-3 | | 130 | 51.2 | 118.0 | 0.071 | 0.8 | 4339 |
| | A-4 | | 145 | 51.2 | 108.0 | Not measured | 0.9 | 4741 |
| | B-1 | 3.0 | 95 | 52.0 | 199.7 | 0.069 | 33.4 | 2604 |
| | B-2 | | 110 | 51.4 | 179.7 | 0.071 | 17.4 | 2860 |
| | B-3 | | 120 | 48.1 | 139.7 | 0.070 | 14.2 | 3443 |
| | B-4 | | 130 | 46.8 | 141.3 | 0.076 | 2.3 | 3312 |
| Comparative | 2 | | 145 | 42.0 | 154.7 | Not measured | 0.9 | 2715 |
| Example | | | | | | | | |
| Comparative | 3 | 120 | 80 | 51.0 | 246.3 | Not measured | 37.5 | 1473 |
| Example | | | | | | | | |
| Comparative | 4 | 30 | 90 | 44.5 | Not measured | 0.162 | Not measured | N/A |
| Example | | | | | | | | |

The above mirin tastes similar to those produced by a conventional method. Thus, the sugar solution of the present invention can be suitably used for producing mirin.

(Discussion) The results clearly show that the sugar solution of the present invention can significantly reduce the amount of the sediment to be generated in mirin when the sugar solution is used as a raw material of the mirin, compared with conventional sugar solutions.

The above also clearly shows that the sugar solution of the present invention can be produced highly efficiently by the production method of the present invention.

### [Example C-1] Producing Sugar Solution, Using KID's Cooker (Apparatus for Heating and Sterilizing Liquid)

(Materials and methods) Anon-glutinous rice flour was used as a raw material.

A raw material rice, liquefying enzyme (α amylase: α amylase 800 (made by HBI Enzymes Inc.) and hot water were introduced into a mixing tank, and the mixture was heated to 60° C. to perform liquefaction (emulsification). The mixing ratio of hot water to rice (Volume ratio) was 110 of hot water to 100 office. The amount of the liquefying enzyme added was 1/3,300 based on the weight of rice.

The emulsification was performed for 10 minutes. The viscosity of the resulting emulsified solution (slurry) was about 500 cps.

The above liquefied solution (emulsified solution) produced was introduced into a reactor for saccharification (KID's Cooker made by Okawara Mfg. Co., Ltd.) through the loading port to carry out saccharification. Vapor was blown into a double jacket tube so that the temperature in the reaction tube was 110° C. to heat the reaction system of saccharification indirectly from the surroundings. The reaction was performed while transferring the liquefied solution to the discharge port from the loading port of the reactor with stirring. The temperature in the reaction tube was kept at the above predetermined temperature during the transfer.

The reaction time (digestion time) was 1.5 minutes.

(Evaluation) The amount of extract components (% by weight) was measured by the Official Analysis Method of the National Tax Agency.

(Results) The results are shown in Table 7. As shown in the table, a sugar solution containing a large amount of an extract component was produced by the production method of the present invention using KID's Cooker.

The temperature of heating and the time of treatment for the inventive product of this Example are the same as those used in Example A-1, and only the heating instrument was different. Thus, the amount of soluble protein generated by heating was equivalent to or a little smaller than that in Example A-1 (52.6% by weight). Accordingly, the amount of soluble protein is also smaller than that in Example A-1 (121.6 μg/ml).

TABLE 7

| | | Time of | Temperature of | Amount of |
|---|---|---|---|---|
| | | digestion | digestion | extract component |
| Category | No. | [minutes] | [° C.] | [% by weight] |
| Reference | C-1 | 1.5 | 110 | 48.2 |
| Example | | | | |

### INDUSTRIAL APPLICABILITY

The present invention provides an industrially useful sugar solution in which the amount of soluble protein is small and the amount of extract components is large, a seasoning and food and drink using the same, and a method for producing them. Accordingly, the present invention greatly contributes to the development of seasoning industry, food and drink industry, and related industries.

### REFERENCE SIGNS LIST

1 Mixing tank
2 Transfer-type mill
3 Reactor for saccharification
4 Loading port of reactor for saccharification
5 Discharge port of reactor for saccharification
6 Holding pipe for holding for reaction time
7 Evaporation tank
8 Buffer tank

1. A sugar solution comprising water and a reaction product of rice and α amylase in the water, wherein the amount of soluble protein in the sugar solution is 200 μg/ml or less and the amount of an extract component in the sugar solution is 43% by weight or more based on the total amount of the sugar solution, and wherein
a reaction for preparing the reaction product of rice and α amylase is a reaction performed at a temperature of 90° C. to 150° C. for 1 minute to 3.5 minutes, with blowing water vapor into a liquefied solution or an emulsified solution prepared from rice, α amylase and water, and
the time of liquefaction for preparing the liquefied solution or the emulsified solution from rice, α amylase and water is 15 seconds to 30 minutes and the temperature of liquefaction is 55° C. to 80° C.

2. A sugar solution prepared by further concentrating and/or storing the sugar solution according to claim 1, wherein the amount of soluble protein in the sugar solution is 200 μg/ml or less and the amount of an extract component in the sugar solution is 43% by weight or more based on the total amount of the sugar solution, and the concentrating is concentrating of the sugar solution to a volume of not less than 90% of the volume of the sugar solution before concentration and the storing is storing at 55° C. to 70° C. for 20 minutes to 22 hours.

3. The sugar solution according to claim 1 or 2, wherein the rice comprises non-glutinous rice or flour of rice for beer.

4. The sugar solution according to any one of claims 1 to 3, wherein the sugar solution is used for producing a seasoning, or a food and a drink.

5. The sugar solution according to claim 4, wherein the seasoning or food and drink is mirin.

6. A seasoning or a food and a drink, prepared by mixing the sugar solution according to any one of claims 1 to 4.

7. The seasoning or food and drink according to claim 6, wherein the seasoning or food and drink is mirin.

8. A method for producing a sugar solution, comprising performing a reaction at a temperature of about 90° C. to about 150° C. for about 1 minute to about 3.5 minutes, with blowing water vapor into a liquefied solution or an emulsified solution prepared from rice, amylase and water to give a reaction product as a sugar solution, in which the amount of soluble protein is 200 μg/ml or less and the amount of an extract component is 43% by weight or more based on the total amount,
wherein the time of liquefaction for preparing the liquefied solution or the emulsified solution from rice, amylase and water is 15 seconds to 30 minutes and the temperature of liquefaction is 55° C. to 80° C.

9. A raw material for the sugar solution according to any one of claims 1 to 5, in which the raw material is a reaction product of rice and amylase, wherein the value of (the amount of the extract component in the reaction product [% by weight])/(the amount of the soluble protein in the reaction product [w/v %]) is 2,000 or more.

10. A sugar solution in which the amount of soluble protein is 200 μg/ml or less and the amount of an extract component is 43% by weight or more based on the total amount, and the volume of a sediment before removing the sediment is 35 or less, wherein the volume of the sediment is a ratio of the height of the sediment to the height to the surface of the solution (%), the sediment being produced by pouring the sugar solution into a container having a uniform shape in a longitudinal direction, together with an aggregate, and stirring the mixture and then allowing the mixture to stand indoors for 3 days.
